(19) United States
(12) Patent Application Publication
(10) Pub. No.: US 2025/0066845 A1
(43) Pub. Date: Feb. 27, 2025

(54) Early Detection of Cancer, Recurrence Monitoring and Companion Diagnostics via Liquid Biopsy

(71) Applicant: Bahram Ghaffarzadeh Kermani, Los Altos, CA (US)

(72) Inventor: Bahram Ghaffarzadeh Kermani, Los Altos, CA (US)

(73) Assignee: Crystal Genetics, Inc., Los Altos, CA (US)

(21) Appl. No.: 17/494,632

(22) Filed: Oct. 5, 2021

Related U.S. Application Data
(60) Provisional application No. 63/088,131, filed on Oct. 6, 2020.

(51) Int. Cl. C12Q 1/6869; C12Q 1/6886; G16B 20/00; G16B 30/00; G16H 50/20

(52) U.S. Cl. CPC C12Q 1/6869; C12Q 1/6886; G16B 20/00; G16B 30/00; G16H 50/20

(57) ABSTRACT

Detection of cancer at an early stage is of great importance, as it significantly improves the odds of a successful outcome for the patient. Similarly recurrence monitoring and companion diagnostics are important, as they could help with identifying the appropriate response to a cancer. The main focus of this invention is on early detection of cancer for screening applications using the non-invasive liquid biopsy methods.

The present application claims priority from the U.S. Provisional No. 63/088,131 entitled “Sequencing of Targeted Biological Material for Early Detection of Cancer, Recurrence Monitoring and Companion Diagnostics via Liquid Biopsy,” filed Oct. 6, 2020, the entirety of all of which are expressly incorporated herein by reference.

### TECHNICAL FIELD

This disclosure generally relates to using liquid biopsy for early detection of cancer, recurrence monitoring, and companion diagnostics.

### BACKGROUND OF THE INVENTION

Regardless of their modality, liquid biopsy methods for detection of cancer from solid tumors share the challenge of having to deal with a small number of molecules. This is where tissue-based assays have access to several orders of magnitude higher numbers of molecules, and therefore are significantly simpler to process and analyze. Nevertheless, liquid biopsy is less invasive for the patient to experience, and more convenient for the clinician to offer. For liquid biopsy to work, however, the information from the molecules-of-interest must be obtained and integrated. Each liquid biopsy assay has its unique challenges. These challenges, in turn, take their toll on the quality metrics such as false positives, false negatives, and limit of detection (LoD). While it is simple and somewhat customary to trade off one metric for another (e.g., lower false positives at the expense of higher false negatives), a clinical application demands simultaneous optimization of all these quality metrics. In other words, any compromise to any of these metrics would translate to compromising care for the patient. Here, we propose a mechanism, by which these metrics can be simultaneously optimized. The crux of this methodology relies on performing high-quality analysis on single cells obtained by high-quality assays. Working with single cells has its unique challenges, from identification and extraction of rare cells, to amplification of their genomic material, and bioinformatics processing of the resulting data. While the single-cell assay described in this invention is significantly more complex than the ordinary liquid biopsy assays, it provides a unique opportunity for enabling high-end solutions including early detection of cancer, recurrence monitoring, and companion diagnostics (CDx).

### BRIEF DESCRIPTION OF THE DRAWINGS

FIG. 1 shows the main proposed single-cell analysis architecture.

FIG. 2 shows an example of a Computer System.

FIG. 3 shows an example of a DNA Sequencing System.

### DETAILED DESCRIPTION

Single Targeted Cells (STCs) such as in Circulating Tumor Cell (CTC) technologies have been used in the past. However, this usage has historically been limited to prognosis and treatment options for advanced (e.g., metastatic) cancers [10]. The critique on CTCs for early detection has been mostly on its sensitivity. The main focus of this invention is to increase the sensitivity of an STC-based (and particularly a CTC-based) system, so it would be appropriate for early stage cancer screening applications. The second focus is on the improvement of the specificity, as many STC-based (and mostly CTC-based) systems are known to extract not only cancerous cells but also benign cells [11], or other cells of no interest.

Specificity is also in terms of recognizing one type of cancer from another. For instance, a test for breast cancer should not become positive if the patient has liver cancer. A liver cancer test should. It is important for cancer tests to be specific to a particular cancer type, as it would not require the tissue-of-origin (Tisor). In a blood-based test, identifying the tissue-of-origin is necessary, as otherwise, the follow-up examinations could become complex. The ideas of this invention could hold for cancer-specific (with tissue-of-interest detected) as well pan-cancer tests, although the main focus would be on cancer-specific tests.

It can be shown that an early detection (of cancer) test for population-wide screening should have very high specificities, e.g., in the 99.99% (as opposed to e.g., in the 99%) range. In order to achieve such high specificities, an STC extraction, by itself, is not sufficient. In this invention, a next-generation sequencing (NGS) operation is suggested to follow the STC extraction (and potentially purification). It can be shown that if the STC extraction and the NGS process each have a specificity of 99%, the overall specificity would be 99.99%, and therefore enable a solution for population-wide screening.

The methods employed in this invention also lend themselves to Companion Diagnostics (CDx) applications, in which specific loci are interrogated with high sensitivity and high specificity, in the search for the variant of interest, which normally has a clinical significance.

### Sources of Single Cells

Throughout this invention, Circulating Tumor Cells (CTCs) are used as a representation of STCs. These are normally the cells that flow in circulation, and have originated from a solid tumor. However, CTCs can be in the form of a cluster of cells, i.e., a group of single cells. Also, single cells could come from different sources than blood, for example from lymph (or lymph nodes), skin, hair, pap smear, buccal smear, saliva, urine, or stool. In general, in this invention, the term Liquid Biopsy is meant to represent the methods of obtaining molecules-of-interest (e.g., CTCs) from sources other than the tissue itself, via methods that are not as invasive as taking a tissue biopsy, e.g., blood, lymph and other sources mentioned above. The method of collecting single cells could also vary, and could comprise the following methods:
1. Natural discharge (e.g., saliva, urine or stool);
2. Irregular discharge, e.g., from breast;
3. Smear, e.g., pap smear, buccal smear;
4. Fine needle aspiration (FNA), e.g., from breast or thyroid;
5. Regular needle aspiration, e.g., aspiration of blood from veins, cerebrospinal fluid (CSF), or lymph from lymph nodes; or
6. Other extraction methods, e.g., bone marrow.

It must be noted that although many of the examples in this invention are on CTCs, the single cells do not necessarily need to have come from circulation (blood); and they could also come from any of the sources that were mentioned above. Also, the circulating cells could be of tumor origin or immune system origin. In the latter case, the immune system cells could be taken as a proxy for cancer cells (since they could be indirectly correlated with the cancerous cells).

In all embodiments of this invention, it is assumed that a sample is collected using any of the above methods, and then the sample is enriched for the STCs. The STCs are then processed to identify markers that could differentiate the state of cancer in the individual, or the tissue-of-interest in that individual. In this regard, the STCs could be directly related to cancer (e.g., representing a cell that is from cancerous origin).

Alternatively, the STCs could be indirectly related to the cancer (e.g., representing an immune cell that has been triggered in response to cancer).

Conventional CTC extraction methods

The CTC extraction methods are conventionally done in two different ways:
1. Using proteomics markers. The antibody affinity targeting certain markers that may be on the tumor cells, or the tumor cells of interest (e.g., in breast cancer)
2. Using physical properties: In this method, CTCs are separated from the normal cells using physical properties such as size, roundness, etc.

Regardless of the extraction method, CTCs can be also labeled and separated using flow cytometry or similar methods.

### Prior Cancer

The application of this invention comprises both cases of de novo cancer (i.e., no past history of cancer) or recurrence monitoring (i.e., compared to a previous cancer). In both cases, the invention holds as it relates to the detection of a cancerous state.

### General Cell Enrichment Strategies

This invention has two main cell-enrichment (cell-separation) strategies that differ slightly on the form of purification of the STCs. The claim sets explain each of the two strategies.

In both Strategies, the cancer signature detection may rely on using tumor-normal pair data. Strategy 1 does STC purification via physically separating single STCs. Strategy 2 does STC purification via growing a culture in which STCs over-multiply as compared to the regular cells, and therefore after a certain period of time, the culture is expected to have more cells from the tumor cells than normal cells [13]. Not every cell has this property. But, certain cells do such as invasive CTCs.

Most of the descriptions in this document are on Strategy 1. However, it should be noted that the methods are applicable to Strategy 2 as well, with the main difference being the step(s) of cell separation/purification.

### Subjects

In this invention, the subject is the member of the species to whom this invention is applied. Primarily, the subject is a human subject. But, without a loss of generality, the concepts could be applied to other species as well.

The subject at the time of the testing could be any of the following:
1. Not having a condition (herein referred to as healthy or normal)
2. Having a condition, such as cancer but not know about it, and hence the need for screening
3. Having a condition, such as cancer, and knowing it, but seeking (directly or via clinician) a way or ways to best treat the condition, and hence the test.

### Biological Sample

A biological sample (also referred to as biospecimen) is the sample obtained from the subject. Biological samples come in different forms, comprising the following:
1. Whole blood, from the subject
2. Plasma, extracted from the blood of the subject
3. Serum, extracted from the blood of the subject
4. Other blood-derived samples from the subject
5. Urine sample from the subject
6. Saliva sample from the subject
7. Buccal swab sample from the subject

In this document Biospecimen and Biological Sample are used interchangeably.

### Sample Acquisition

The sample (e.g., blood) is acquired from the donor/patient. For identifying CTCs in blood, in the best mode, between 5 mL and 50 mL of blood would be needed. Higher volumes of blood result in better CTC counts. However, higher volumes may pose economical and ethical limitations.

### Condition of Interest (Col)

The condition of interest is the condition that this invention seeks to identify. For instance, the condition is a disease, and the disease is cancer, and the cancer is breast cancer.

In general terms, however, Col could refer to a condition that may not necessarily be considered a disease, for instance it could be a benign condition, e.g., a benign cyst. Col could also be a pre-disease, for example pre-cancer, e.g., Ductal Carcinoma In Situ (DCIS) of breast.

In the case of a disease, the Col may be referred to as the Disease of Interest (DoI).

### Feature(s) of Interest (FoI)

A feature of interest (FoI) is an individual entity which carries the information which would be useful for the analysis, which usually is performed in relation to the Condition of Interest. Examples of FoI are CTCs or Exosomes.

Mostly, the features of interest have information about the Condition of Interest, e.g., cancer. However, FoI could also include features that do not have a direct information value, but rather have an indirect informational value, for example features (e.g., cells) that are used/isolated for normalization purposes, e.g., while blood cells (WBCs) or subtypes of WBCs such as T-Cells or B-Cells. To avoid confusion, we may also refer to this latter group of features (for normalization) as Co-Features of Interest (Co-FoI). Nevertheless, we consider Co-FoI as a subclass of FoI, as Co-FoI are also of interest in the analysis. Therefore, the term Features of Interest (FoI) is used to stand for not only Features of Interest but also Co-Features of Interest. In the case of normal cells (e.g., healthy/normal cells such as white blood cells), they can be obtained from the enriched cells, or from the original blood.

FoI could also include the Region of Interest (as described below). In this case, for example, instead of looking for cancerous cells, the FoI would be cancerous cells from the breast.

Therefore, the definition of the FoI has to do with the Col. If the Col is only detection of cancer, then the FoI could be cancerous cells. If the Col is the detection of breast cancer, then the FoI could be cancerous breast cells.

Therefore, in a general form, FoI could include any feature that is related to detection, diagnosis, screening, prognosis, etc. in relation to the Col.

### Feature Types and Subtypes

The types of features could vary. Below are certain examples of these feature types:
1. Cells 2. Exosomes

Cells can have different subtypes, based on various features.

Throughout this document, the term cells are used as the feature of interest. However, it must be noted that similar arguments could be applied to exosomes.

One of the main differentiating factors in cell analysis is the form of the cell. The following are some examples:
1. Live cells: The cells that still function as functional cells, whether their function is normal (as in healthy cells) or abnormal (as in cancerous cells).
2. Fixed-only cells: The cells that are treated with fixatives, such as 2% Paraformaldehyde (PFA) or in special-purpose tubes containing certain fixatives (e.g., CellSave preservative tubes from Menarini Silicon Biosystems [23]).
3. Permeabilized-only cells: For example cells that are permeabilized using Triton-X 100).
4. Fixed and Permeabilized cells: Cells that are fixed to retain their structure, and then permeabilized (e.g., using 0.2% Triton-X 100).

While possible with live cells, much of the cell fluoroscopy and also cell picking is done using fixed and permeabilized cells. Also, certain biomarkers (such as cytokeratin) only work when the intracellular space of the cell is available, which means the cells would have to be permeabilized.

An assay may be done under the following scenarios (among other possible scenarios):
1. All cells (live and dead)
2. Live cells
3. Cells that include fixation (CIF) (e.g., fixed-only, permeabilized-fixed)
4. Live cells followed by CIF:
   a. Where the live cells are different than CIF
   b. Where live cells and CIF are the same cells, e.g., assay is first done on live cells, and then the cells are permeabilized-fixed, and then the rest of the assay is done on the same cells which are now in a fixed state.
5. Dead cells, where the dead state could be due to any reason, including fixation.

### Feature Enrichment

As a building block in the core of this invention, there is an enrichment step, where the FoI are preserved (as much as possible) and the features of no-interest (FoN) are suppressed or removed. For instance, if a mix has 1 unit from FOI in the background of 1 million FoNs, after enrichment, a significant fold increase in the contents of FoI is achieved. For instance 1 FoI in 1000 FoNs, which would represent a 1000-fold enrichment.

In reality, the enrichment processes are somewhat lossy, meaning some of the FoIs are lost, as the FoNs are being reduced. The percent of the FoIs that remain after enrichment, compared to prior to the enrichment, is often referred to as Recovery. The recovery could vary (for example) between 50% and 100%. In the best case of Recovery=100%, no FoI is lost. In the case of Recovery=50%, half of the FoIs are lost in the process of enrichment.

Examples of Feature Enrichment (in particular CTC enrichment) systems are provided in [28], [26], and include the following: CellSearch, ISET, MetaCell, ScreenCell Cyto, CellSieve, Parsortix, RosetteSep, OncoQuick, Cyttel, AccuCyte-CyteFinder, EPISPOT, Vita-Assay, CytoTrack, FASTcell, ImageStream, Vortex, ClearCell, DynaBeads, GILUPI CellCollector, IsoFlux, MagSweeper, MagDense, MetaCell, NanoVelcro, pluriSelect, Reactive Ion Etching, TelomeScan, GEDI, OncoCEE, Herringbone Chip, and Magnetic Sifter.

Feature Enrichment can be done via Negative Enrichment, Positive Enrichment, or a combination of them. For example:
1. Negative Enrichment only
2. Positive Enrichment only
3. Negative and Positive Enrichment together
4. Negative Enrichment followed by Positive Enrichment
5. Positive Enrichment followed by Negative Enrichment

### Negative Selection

Blood itself is made up of many cells, e.g.,:
Erythrocytes (RBC)
Platelets
Reticulocytes
Neutrophils (granulocytes)
Lymphocytes
Monocytes
Eosinophils (granulocyte)
Basophils (granulocyte)
CD34+ cells
CEC (circulating endothelial cells)
CD34+ CD38− cells
EPC (endothelial progenitor cells)

In blood, cells from other tissues can also be found, for example mammary cells (from breast). These cells could be normal, benign or cancerous. What is known as circulating tumor cell (CTC), could belong to a tumor that is of benign or cancerous origin.

CTCs are the main cells of interest for this application. Other cells can be considered either as noise or having use in normalization. Much of the hematopoietic-origin (blood-based) cells fall into this category. For instance leukocytes (while blood cells). Many of these cells express proteins such as CD45 on their surface. Therefore, depletion of the CD45-positive (CD45+) cells should result in a reduction of noise in the system, and make the search for CTCs easier. (The CD45 antigen is expressed on all hematopoietic cells except erythrocytes and platelets.)

The task of reduction can be done by two types of processes:
1. Negative Selection
2. Depletion

Technically, in Negative Selection, cells that do not express a certain marker are identified and pulled out. This may be done by identifying markers that are exclusive (in nonexpression) to the cells of interest, pulling out those cells, and therefore arriving at a negatively selected population. In Depletion, the targeted cells are pulled out (depleted) from the mix.

In this application, without a loss of generality, we will use the terms negative selection and depletion interchangeably, i.e., to denote their function. Other depletion technologies or other variations of these technologies can also be utilized in this invention. Among methods for performing Depletion, the following commercial kits can be mentioned as examples: pluriSpin by pluriSelect [25], and EasySep/RosetteSep by Stemcell Technologies [24].

### Types of Negative Selection

Negative selection can be done in two different ways:
1. Physical Negative Selection (PNS)
2. Informatic Negative Selection (INS)

In the first method (PNS), the cells are physically filtered out, and do not proceed to the next level of processing. For instance, hematopoietic cells can be removed using their markers.

PNS can in turn be divided into a few categories including the following:
1. Centrifuge-based methods (such as spinning blood to separate red blood cells (RBCs) from the rest.
2. Density gradient methods, such as separation (via centrifuge) using Ficoll, Percoll, or another density gradient medium (DGM).
3. Affinity-based method: In this method, the features of interest (for negative selection) are involved with another entity via an affinity mechanism, e.g., antibody-antigen based, or another type like biotin-streptavidin based. Examples of antibody-antigen-based Affinity-based selection are finding CD45+ as antigen expressed on/in the cell (or CDxx where xx=1 to 999, or cells with other antigens on them) features/cells using anti-CD45 antibodies which may be conjugated to a bead (e.g., as in pluriSpins) or to another entity such as RBCs (via tetrameric antibody complex), for example as RosetteSep [26], [27]. Affinity could be in the form of binding.

In the second method (INS), the cells are marked (e.g., via immunostaining) and the marked cells are consequently omitted (informatically) from the rest of the process. For instance, the cells that express CD45 are stained with anti-CD45 antibody either conjugated to a dye directly, or via a secondary (or secondary+tertiary) antibody which is conjugated to a dye. Once stained with the color of the dye of interest (e.g., red) those cells that are colored are not considered in the rest of the processing. For example, they are not labeled as potential candidates of the cells of interest (e.g., cancer cells). In this manner, they marked cells, here, are effectively removed from the processing, and hence the operation “informatic” negative selection (INS) is done on them.

In either PNS or INS method, the negative selection could be based on 1 label (e.g., stain) or multiple labels (e.g., 2 or more stains). For instance, an undesired cell (subject of negative selection) could be represented by 1 or more fluorescent dyes. The type of marker (for detection) could also be generalized beyond fluorescent staining, e.g., it could be using other modalities like radioactive marking.

Similarly, in either PNS or INS, a certain dye could be used to label 1 or more undesirable cells (subject of negative selection). For instance, both CDxx and CDyy cells (where xx and yy could be 1 through 999), or any other antibody that is subject to elimination could be represented by the same (single) dye, for example the color “red.” While of course, in this case, color red cannot necessarily distinguish between CDxx and CDyy, it can be used to state that at least one of CDxx and CDyy have been expressed on the cell(s) of interest, and therefore it can be eliminated (via negative selection). A combination thereof can also be used, where some have 1 or more labels, and 1 or more labels could correspond to one.

### Positive Selection

Positive enrichment is done by either selecting the cells of interest directly, e.g., via antibody-antigen interaction, or by negative selection of all/many/most of the cells, except the cell of interest, or cells of interest.

### Types of Positive Selection

Similar to negative selection, a positive selection can also different methods:
1. Physical Positive Selection (PPS)
2. Informatic Positive Selection (IPS)

In the first method (PPS), the cells of interest (for positive selection) are physically pulled out (extracted and passed for further processing) or enriched among other cells.

In the second method (IPS), the cells of interest are labeled, e.g., via an antibody which is conjugated to a dye either directly or via secondary/tertiary antibody systems. Similar to the arguments on the negative selection, the IPS can be done via 1 or more dyes per feature of interest. Alternatively, multiple markers (e.g., EpCAM and EGFR) can be labeled in a similar way, e.g., via the same staining (dye) or an enrichment mechanism (e.g., magnetic). A combination thereof can also be used, where some have 1 or more labels, and 1 or more labels could correspond to one.

Throughout this document, we use the term Positive Selection and Positive Enrichment interchangeably. Both are meant to enrich the cells of interest. The enrichment could be any positive number greater than 0% and less than 100%, although normally enrichment is referred to making the proportion 50% or higher.

While Positive Selection normally involves direct selection of the cells of interest, it could also be implemented in the other methods including the following:
1. Selection of certain entities, where those entities, directly or indirectly, select/enrich the cell(s) of interest.
2. Selection of many/all other cells (but the cells of interest) and eliminating them (physically or informatically) from the process. For example, to enrich CTCs in blood, many of the hematopoietic cells could be eliminated.

### Combination Selection

When selection is made based on a threshold on a measured observation, the distinction between positive and negative selection may be subtle and subject to interpretation. For example, assume size as the feature of interest. In other words, assume cells with a size greater than a threshold are the cells of interest. In this case, when the size of the cell is estimated, one could reject a cell if it is smaller than the threshold, or keep it if it has the size equal to or greater than the threshold. It is unclear whether this is particularly a negative selection or a positive selection.

In this case, one could call this method a combination selection. A combination selection could be considered a negative selection or a positive selection, depending on the interpretation, such as in the above example. In the rest of this document, we may not specifically mention Combination Selection. However, it should be noted that a negative selection could include Combination Selection. Similarly a positive selection could also include a Combination Selection.

Combination Selection could be informatic or physical. For instance, in the above example, if the size is measured via staining/imaging, then it is informatic. However, when the elimination/selection is done via physical means like sieve of some sort, Combination Selection is Physical.

In any case, when the source of selection is physical, we usually reserve the term “physical” to describe that situation.

### Feature Identification

A FoI can be identified using different modalities, including microscopy. In microscopy (such as in fluorescent microscopy), the FoI or FoN are stained using different biomarkers.

The staining methods comprise the following:
1. A primary antibody, conjugated to a fluorescent dye
2. An unconjugated primary antibody, and a secondary antibody (targeted to the said primary antibody) conjugated to a fluorescent dye
3. An unconjugated primary antibody, and an unconjugated secondary antibody (targeted to the said primary antibody), and a tertiary antibody (targeted to the secondary antibody) conjugated with a fluorescent dye.

The staining can also be done using a combination of an unconjugated antibody, but via biotin-streptavidin interaction. For instance: A primary antibody conjugated to biotin, and a fluorescent dye conjugated to streptavidin. Other such molecule-to-molecule or molecule-to-marker interactions are also allowed.

### STC/CTC Identification

Category 1: Cell biomarkers. Examples:
A. Positive for Epithelial cell adhesion molecule (EpCAM+)
B. Positive for EGFR
C. Positive for Cytokeratin (CK+)
D. AND or OR of the above operations.

Category1 can be broken down into multiple Subcategories:
A. Subcategory 1A is for enrichment of CTCs using some cell markers. Examples of these antibodies are EpCAM and EGFR.
B. Subcategory 1B is for confirmation (increasing specificity). In this stage a set of antibodies are used. These antibodies vary depending on the use case. For breast cancer (of epithelial origin), some Cytokeratin antibodies could be used in order to select for the cells that are of epithelial origin.

Each of these subcategories can be done for live or fixed cells (often also permeabilized).

Category 2: Biomarkers for differentiating STC/CTCs from regular cells in the blood (e.g., white blood cells)
A. Negative for leukocyte common antigen (CD45−) to differentiate CTCs from white blood cells
B. Positive for having a nucleus (Nucleus+/DAPI+) to identify nucleated (viable) CTCs, and also to differentiate CTCs from residuals of regular red blood cells (RBC)
C. Positive for physical properties, such as larger than average size for STC/CTC vs. regular cells.
D. A and B
E. A and B and C

Category 3: Biomarkers for identifying complementary states of the cells.
A. where the complementary state is the mesenchymal state of the cells, e.g., for the cells that have undergone an epithelial-to-mesenchymal transition (EMT) [7]. Examples of mesenchymal markers are Vimentin (VIM) or Slug [7].
B. where the complementary state is a biomarker that is semi-specific to a subset of cancer disease
   a. Where the disease is breast cancer
      i. Where the markers are HER2, Estrogen (ER) and Progesterone (PR)
b. Where the disease is prostate cancer
   i. Where the marker is Prostate Specific Antigen (PSA)
c. Where the disease is lung cancer
d. Where the disease is colorectal cancer
e. Where the disease is head and neck cancer
f. Where the disease is a virus-derived cancer
   i. Where the virus-derived cancer is due to Human Papillomavirus (HPV)
   ii. Where the virus-derived cancer is due to Epstein-Barr Virus (EBV)
   iii. Where the virus-derived cancer is due to Hepatitis C Virus (HCV)
C. A and B

### Feature Isolation Step (FIS)

After identification, the feature of interest could be isolated/picked for further analysis. In this step, each feature (e.g., a CTC) is picked or otherwise isolated from the others. The isolation can be done in separate hard units (like tubes) or soft units (like emulsion units, similar to those used in emulsion PCR).

The isolation can be done for 1 FoI or a pool of FoIs. For instance, there can be 1 CTC in a tube, or there can be a pool of 10 (or 5-10) CTCs in a tube. The pool of CTCs may or may not include other cells, e.g., white blood cells (WBCs).

Isolation can be done physically or informatically. In physical isolation, the cell(s) of interest is (are) physically picked or otherwise placed in different compartments for further analysis. In informatic isolation, the location of the cell(s) of interest is (are) marked, and the marked entities (cells of interest) are used for further processing. In physical isolation, the act of picking can be done manually (e.g., pipetting) or automatically (or semi-automatically) via an instrument (e.g., a robot), for instance Fluidigm C1 [15], DepArray [15], CellSelector [15], and CytePicker [27].

Among informatic isolation is the option of processing the enriched cells, and then performing the rest of the processing (e.g., next-generation sequencing), and via mapping to the reference genome, identify (informatically) which fragments correspond to which cells. For example, fragments carrying certain mutations may correspond to cancerous or other anomalous cells.

It must be noted that cell picking is only one type of physical isolation. Physical isolation comprises other methods, e.g., placing cell(s) in chambers without physically picking them. Chambers that are used for physical isolation could be solid (like wells, tubes, etc.), or non-solid, e.g., emulsion bubbles, etc.

Isolation could also be non-physical (i.e., informatic) in the sense that the task of isolation could be done by labeling/marking the cell(s) of interest with certain markers (or simply remembering the position of them, e.g., the X/Y coordinates of them on a surface) that could help the identification of the cell(s) of interest in that step and/or the following steps of the process.

### STC Purification

A. Where purification of STC is selecting a single STC from the collection of residual cells including STC and other cells such as white blood cells.
B. Where purification of STC is done using an appropriate STC culture [13].

### Feature Molecular Analysis (FMA)

The isolated feature(s) can then undergo a molecular analysis. This step is called

### Feature Molecular Analysis (FMA).

In its simplest form, FMA could be counting or enumeration of the cell(s) of interest, in absolute terms, with or without normalization to the original amount of the biological material. For instance, it could be 10 cells in the assay; or as another example, it could be 7 cells per mL of the whole blood (or alternatively processed) biological material. FMA could also include the ratiometric measures, e.g., number of cells that are stained with a certain (antibody-) dye vs. all cells, or alternatively vs. cells that are stained with 1 or more different (antibody-) dyes.

FMA includes Analysis or Pre-Analysis steps. Examples of the latter are Nucleotide Amplification (NAM) operations, such as whole genome amplification (WGA). Other steps prior to sequencing are Sample Quality Control (QC) (e.g., using Qubit from ThermoFisher and/or TapeStation by Agilent [33]), purification (e.g., using Ampure

Beads from Beckman Coulter [29]), shearing (sonication or Covaris [32]), library preparation (e.g., to make the DNA molecules contain the adaptors/indexes that are required for sequencing), library QC (e.g., using Qubit and/or TapeStation). Examples of library preparation kits can be found in and include: Nextera, KAPA, NEBNext, TruSeq, and QiaSeq FX. Throughout this invention, the term sequencing is meant to include all the steps of preparing DNA for sequencing, as stated above, starting from Sample QC.

It should be noted that the FMA can be done without physical isolation of the cells. In other words, it can be done by just labeling the cells of interest with specific markers (e.g., dyes) or other methods of marking.

Most commonly, however, in FMA, one or a series of molecular modalities (like DNA or RNA) are interrogated. The interrogation of molecular modalities would depend on the modality of interest. For instance, for DNA, the corresponding analysis could be one or a combination of the below:
1. Next-generation sequencing (NGS)
2. Genotyping, often done by what is called microarrays, gene chips, genotyping chips, or SNP chips. Examples of genotyping chips are Illumina Infinium or Global Screening Array (GSA).
3. Quantitative PCR (qPCR): Small variants can be interrogated using qPCR, with high sensitivity.

NGS itself can be done in various ways, including the following:
1. Targeted sequencing, where only specific regions on the genome are targeted.
2. Whole exome sequencing (WES). This is a special case of the above, where the targeted region is often the exonic regions and splice junctions
3. Whole genome sequencing (WGS), where the whole genome is sequenced, to the extent that is limited by the underlying technology.

The output of FMA can be binary (i.e., Detected vs. Not Detected). It can also be non-binary (e.g., a real number between and including 0 and 1). In the case of non-binary, a Likelihood value can be assigned to FMA. This Likelihood value is used to describe the relationship/correlation between the observed patterns and the Col. For instance, a Likelihood of 0.9 (or 90%) could mean a high chance of the pattern to be from Cancer, hinting that the Subject may have Cancer.

If SQS and FMA are both non-binary, then the overall Likelihood of the Subject having Col can be calculated by combining the two Likelihoods (SQS and FMA). An example of such a combination is to multiply the two Likelihood values. Another example is to calculate the geometric mean of the two values. Other combinations are possible, e.g., arithmetic mean, hyperbolic mean, weighted mean, median, trimmed mean.

### Conversion to Sequenceable Material

Genetic materials (e.g., DNA, RNA) obtained from a small number of cells (e.g., a single cell) are not usually sufficient for sequencing. In other words, an amplification step is usually required, in order to raise the number of molecules so it can fit the requirements of the steps leading to sequencing.
A. Where conversion may comprise an amplification step
   a. Where the nucleotide amplification step is whole genome amplification (WGA)
      i. Where the WGA method is one of the methods mentioned above.
   b. Where the amplification step is amplicon based, targeting certain regions
   c. Where the amplification step includes hybrid capture using a set of probes for targeted regions, followed by a PCR amplification.

### Analysis Mode

Analysis comprises a plurality of methods, including:
A. Where the analysis mode is singleton-based (i.e., using the STC-based sample itself)
B. Where the analysis is Normal Differential Mode (NDM), where the STC sample results are compared to a control/normal sample from the same individual
   a. Where the control sample is from bulk sequencing of normal cells (e.g., white blood cells), where bulk could refer to many cells, or a minimum of two cells). An example of bulk sequencing is sequencing samples from the peripheral blood (which normally contains WBCs).
   b. Where the control sample is from purified single cells of the normal-cell population (e.g., white blood cells)
C. Where the analysis mode is NDM, where the STC sample results are compared to a set of STC samples from other individuals. In other words, a set of samples are used to normalize the sample of interest.
D. Where the analysis is Tumor Differential Mode (TDM), where the STC sample results are compared to a tumor tissue sample from the same individual at the time of a physical biopsy. The TDM's use would be in cancer recurrence monitoring applications, where the profile of the STCs is compared to the general profile of the tumor tissue to see if the STCs are from the same source, i.e., recurrence of the same cancer.
E. Where the analysis mode is TDM, where the STC sample results are compared to a set of STC samples from other individuals. In other words, a set of samples are used to normalize the sample of interest.
F. B and C
G. D and E
H. B, C, D and E

### Whole Sequencing Operation

Sequencing could be applied in a plurality of forms:
A. Where the operation is whole genome sequencing
B. Where the operation is whole exome sequencing
C. Where the operation is a targeted genome sequencing on regions that exist on more than 50% of the chromosomes on the whole genome
D. Where the operation is whole transcriptome sequencing
E. Where the operation is whole methylome sequencing, e.g., via bisulfite conversion.

### Genomic Profile

Genomic profile can be done via somatic and/or germline variants of the corresponding data. Examples of variants are Single Nucleotide Variants (SNVs), Insertions (Ins), Deletion (Del), Block Substitutions, Copy Number Variations (CNVs), Loss of Heterozygosity (LoH), Structural Variations (SVs) including Gene Fusions, or any other known type of genomic variants.

### Disease Claim

The methods described here apply to any Condition of Interest, including a Disease of Interest, and in particular cancer.

### Disease Pattern Recognition

Depending on the results of the previous steps, and in particular the FMA, an association with a disease is established. For instance, it is claimed that the extracted CTCs are from cancerous origin.

This process could use the following elements:
A. To use one or a plurality of signatures to associate the sample as cancer
B. To use one or a plurality of signatures to associate the sample as benign
C. To use one or a plurality of signatures to associate the sample as precancer
D. To use one or a plurality of signatures to associate the sample as normal
E. To use one or a plurality of signatures to associate the sample as unknown (but not normal) condition

Although the best-mode in this invention would be whole genome sequencing (WGS), whole exome sequencing (WES) could be an alternate mode (as well as other potential mentioned methods).

### Signature Detection

A. Where the sequenced data from a singleton sample is compared to known signatures for cancer, to label the STC-derived sample as cancer or non-cancer. Copy Number Variation (CNV) is an example of such signature [9].
B. Where the STC-derived data is compared to the normal cell (bulk or single cell) data (from the same individual) in order to identify the pre-defined differential signatures for cancer, to label the STC-derived sample as cancer or non-cancer. Examples of such signatures are provided in the application U.S. Pat. No. 15,897,152.
C. Where the STC-derived data is compared to the normal cells (bulk or single cell) data (from different individuals) in order to identify the pre-defined differential signatures for cancer, to label the STC-derived sample as cancer or non-cancer.
D. Where the STC-derived data is compared to the tumor tissue (at the time of original biopsy) data (from the same individual) in order to identify the pre-defined differential signatures for cancer, to label the STC-derived sample as cancer or non-cancer.
E. Where the STC-derived data is compared to the tumor cells (bulk or single cell) data (from different individuals) in order to identify the pre-defined differential signatures for cancer, to label the STC-derived sample as cancer or non-cancer.
F. B and C
G. D and E
H. B, C, D and E

### Disease Organ of Interest

The identification of a disease from blood is often not sufficient. One would also have to make a claim or provide a likelihood of that observation to have come from a tissue and/or organ of interest. For example, based on the information from the previous steps, one would conclude that the CTC of interest is most likely from epithelial breast origin.

### Region of Interest

A Region of Interest (RoI) is used to further narrow down the focus of the Fol. For instance, instead of looking for just a CTC (as a FoI), one might need to search for a CTC that is of a particular tissue-type, organ, or both. The specific tissue-type, organ, or combinations-thereof are referred to as Region of Interest. The RoI can be further delineated into the following sub-categories:
1. Organ of Origin
2. Tissue of Origin
3. A combination of the OoO and ToO

In a similar process to the removal of CD45 cells, a system can be set up to remove cells from other origins, as compared to the tissue of interest. For interest, in a search for cells from the breast, one can devise a cocktail that removes cells from blood, lung, liver, etc. origin.

RoI (Organ or Tissue of Origin) can also be done via a positive selection, by searching for antibodies that are most expressed in a certain tissue, for instance by targeting antibodies that are expressed explicitly (or almost explicitly) in a particular cancer, e.g., breast cancer or prostate cancer.

The search for RoI can be done via live cells, fixed cells, or a combination thereof.

Throughout this document the term fixation is meant to represent any of the following, with the second option being the more popular option:
1. Fixation only
2. Permeabilization and fixation

Different fixations can be used, depending on the level of penetration (into the cell) that is needed, for instance if the access to cytoplasm is needed or if the access to nucleoplasm is needed (e.g., in transcription factors).

### Organ of Origin (OoO)

A feature of interest may be further limited to an organ. For example, one may not only be interested in a CTC, but a CTC that has originated from Breast, implying a potential suspect for breast cancer. In this case, the Organ of Origin (OoO) is breast.

Different markers exist that could either fully or partially identify the OoO. For instance, when there is a protein that is expected to only/mostly express in breast tissues, the antibodies that bind to this protein could be used as markers for OoO in breast cancer.

Examples of these markers exist in several organs, e.g., PSA in prostate cancer.

### Tissue of Origin (ToO)

Aside from a particular organ, sometimes, a general tissue type can be associated with Fol. For instance, one may be interested in CTCs that are of Epithelial origin. In this case, epithelial tissue would be the Tissue of Origin (ToO).

There are protein markers that can assist in associating a FoI with a ToO. For example, many subtypes of Cytokeratin can be used to identify epithelial cells. Since these (epithelial) cells are the source of many cancers (e.g., in several breast cancers), then identifying them could assist in associating a FoI (like a CTC) with the corresponding ToO, which would increase the specificity of the assay.

### Extensions to the Core Invention

### Extension to Exosomes

Exosomes are small vesicles formed in vesicular bodies with a diameter of 30-100 nm and a classic “cup” or “dish” morphology. They can contain microRNAs, mRNAs, DNA fragments and proteins, which are shuttled from a donor cell to recipient cells. Exosomes secreted from tumor cells are called tumor-derived (TD) exosomes [19].

Tumor-derived or tumor-associated exosomes contain abundant biological contents resembling those of the parent cells along with signaling messengers for intercellular communication involved in the pathogenesis, development, progression, and metastasis of cancer. As these exosomes can be detected and isolated from various body fluids, they have become attractive new biomarkers for the diagnosis and prognosis of cancer [20].

In another embodiment of this invention, the step of purifying/identifying CTC can be replaced by the step of extraction of tumor-derived exosomes. After such extraction, the resultant biological material (e.g., DNA or RNA) can be taken through the other mentioned steps of the invention, i.e., the steps comprising WGS/WES of the DNA followed by the identification of the cancer signatures in the resultant signals. For a person familiar with the art, this constitutes a natural extension/modification of the main invention which has been described mostly as CTC-centric in this application. Similar to CTCs, the analysis of tumor-derived exosomes can be done in a singleton (tumor-only) mode, or pairwise/differential (tumor-normal) mode.

In yet another embodiment of this invention, the step of isolation of biological material (e.g., DNA or RNA) from tumor-derived exosomes could be replaced by isolation of biological material from the whole exosomes (and not tumor-targeted exosomes). However, since this whole exosome population would have more impurity (i.e., exosomes from normal cells), the resulting signals are expected to have a less signal-to-noise ratio (SNR) as compared to the former method (tumor-derived exosomes).

### Extension to Other Nucleic Acids

While most of the focus of this invention is on DNA, many of the methods are available for RNA and other nuclear and/or cellular entities. In general, Nucleic Acid Analysis (NAA) applies to different types of analyses on elements such as DNA, RNA, etc.

The type of Analysis includes Next-Generation Sequencing (NGS), SNP Genotyping, other Genotyping modalities, real time PCR, quantitative PCR, Fluorescent In-Situ Hybridization (FISH), as well as other methods. NGS includes Whole Genome Sequencing (WGS), Whole Exome Sequencing (WES), and Gene Panel Analysis.

### Extension to Other Sequencing Methods

The core of this invention is on whole genome sequencing. However, the idea can be extended to other sequencing modalities such as whole exome sequencing, targeted sequencing of a small (1 to tens), medium (tens to hundreds) or large number (hundreds to thousands) of genes.

### Extension to Other Interrogation Methods

While this invention mostly focuses on next-generation sequencing (NGS), its applicability is not limited to NGS. For example, genotyping chips (such as Illumina Infinium or Global Screening Array (GSA)) or array Comparative Genome Hybridization (CGH) can be used to find copy number variations. Also, genotyping chips can be used to find selected variants (at the determined loci of interest on the chip).

Similarly, quantitative PCR (qPCR), a.k.a., real time PCR methods can be used to interrogate variations at certain loci. The loci could be point-loci (e.g., SNV or InDel) or global (e.g., Alu).

### Extension to Late Stage

While the essence of this invention is in early stages of cancer (Stages 1 and 2), the methods are also applicable to the late stages (Stages 3 and 4) as well as pre-cancer (Stage 0) cases. In particular, certain applications such as CDx often have most applicability in the late stages of cancer.

### Exemplary Embodiments

Among many embodiments that could be obtained by combining the steps identified earlier in this invention, the following can be mentioned:

### Example Embodiment 1: Pos, Pick, Wga, Ngs

1. Perform positive selection (e.g., by finding cells with antibodies of interest)
2. Identify the cells of interest
3. Pick the cells of interest (e.g., manually or using micro-manipulation)
4. Perform Whole Genome Amplification (WGA)
5. Perform DNA sequencing and/or genotyping and/or qPCR on the WGA product
6. Identify the patterns of interest by looking for the variants of interest

### Example Embodiment 2: Neg, Pick, Wga, Ngs

1. Perform negative selection (e.g., by pulling out cells with antibodies of no interest)
2. Identify the cells of interest
3. Pick the cells of interest (e.g., manually or using micro-manipulation)
4. Perform Whole Genome Amplification (WGA)
5. Perform DNA sequencing and/or genotyping and/or qPCR on the WGA product
6. Identify the patterns of interest by looking for the variants of interest

### Example Embodiment 3: Neg, Pos, Pick, Wga, Ngs 1. Perform negative selection (e.g., by pulling out cells with antibodies of no interest)

2. Perform positive selection (e.g., by finding cells with antibodies of interest)
3. Identify the cells of interest
4. Pick the cells of interest (e.g., manually or using micro-manipulation)
5. Perform Whole Genome Amplification (WGA)
6. Perform DNA sequencing and/or genotyping and/or qPCR on the WGA product
7. Identify the patterns of interest by looking for the variants of interest

### Example Embodiment 4: Pos, Neg, Pick, Wga, Ngs

1. Perform positive selection (e.g., by pulling out cells with antibodies of no interest)
2. Perform negative selection (e.g., by finding cells with antibodies of interest)
3. Identify the cells of interest
4. Pick the cells of interest (e.g., manually or using micro-manipulation)
5. Perform Whole Genome Amplification (WGA)
6. Perform DNA sequencing and/or genotyping and/or qPCR on the WGA product
7. Identify the patterns of interest by looking for the variants of interest

### Example Embodiment 5: Enrich, Isolate, Wga, Analysis w/Normal

1. Perform enrichment for the cell population of interest
2. Perform Identification of the cells of interest
3. Perform Isolation of the cells of interest, where 1 or more cells are isolated in separate containers.
4. Perform WGA on the isolated cells
5. Perform DNA molecular analysis on the WGA product and contrast to the molecular analysis of the normal/healthy cells. Identify differential patterns of interest.
6. Compare the differential patterns of interest with those expected from known cancerous samples (in comparison to their paired normal samples) to arrive at a disease state prediction
7. Arrive at a disease detection

### Example Embodiment 6: Enrich, Isolate, Sample Qualify, Wga, Analysis w/Normal

1. Collect whole blood from the patient/subject.
2. Identify Circulating Tumor Cells (CTCs), i.e., cells that are suspect of having been originated from tumors are identified using a CTC-enrichment system as a configurable system, and then configured/customized, as follows:
   a. Using a customized pre-enrichment workflow, for mostly elimination of unwanted cells and/or increasing the ratio of the wanted cells to unwanted cells.
   b. Using a customized enrichment workflow, to maximize the likelihood of capturing cells from the wanted cells. The customization is to ensure best capturing of the wanted cells, while reducing the unwanted cells.
   c. Using a custom-made post-enrichment antibody-cocktail for positive selection of the wanted cells (optimized for the cancer(s) of interest) and negative selection of unwanted cells.
   d. Picking cells from the pool of wanted cells.
3. If no CTCs are found, call the sample negative.
4. If a set of CTCs are found, select a set from them for imaging/picking.
5. Process the set of imaged/picked CTCs using a Sample Qualification System (SQS), which works based on the image-based features such as an Affinity Based Method (e.g., antibody-staining) and physical properties (size/diameter, shape, aspect ratio, irregularity, etc. of the cells). The output of SQS is usually binary (1=Qualified; 0=Not Qualified). However, in other embodiments, the SQS could have a probabilistic nature, and render a value (Probability or Likelihood) that relates to the probability/likelihood of the sample being Qualified, i.e., being from the condition of interest. Here, we use the term Probability and Likelihood loosely in order to simply show a non-binary value that somewhat correlates with the Sample being Qualified to move to the next step or not. In this non-binary case, in one exemplary embodiment, the sample is moved to the next step if the Likelihood of it is above a certain threshold. In another exemplary embodiment, members (e.g., Single Cells, Cell Aggregates/Clusters or Cell Pools) of the Sample that are of high Likelihood are selected and passed to the next stage as Qualified. Here, we use the term “High Quality” for when the Likelihood of an entity (Sample or members of the Sample) is above a predefined threshold. The Likelihood value is a number (e.g., a real number, or a fractional number) between (and including) 0 and 1. Examples of such thresholds are 0.8, 0.9, 0.99.
6. Obtain a binary (Qualified vs. Not Qualified) call from SQS, for the sample. If the output of SQS is 0, the sample is called negative, and no further action is done. If the output of SQS is 1, the sample is passed forward to the next step.
7. If the sample is qualified by SQS, select a set of cells from that sample for further processing. This set is defined by CTCs with the highest likelihood (which may be provided by SQS) to be from the organ/tissue of origin, as well as normal cells. At this step, X cell tubes are selected and passed forward, for further processing.
8. Perform a whole genome amplification (WGA) operation on the X cell tubes that are passed to this stage, resulting in amplified DNA for each of their contents.
9. Quality Control (QC) the DNA corresponding to the cells, in order to assess the success of the amplification process.
10. Proceed the DNAs that pass the QC (if not an empty set) to the whole genome sequencing (WGS) step. If no cell passes QC, the sample will be called NoCall.
11. Sequence the DNAs that passed QC, using a DNA sequencing instrument.
12. Process the raw files of sequencing. Interrogate the WGS data for signatures of cancer (e.g., genome-wide copy number variation) in the patterns of the CTC and normal WGS data.
13. If a cancer signature is not observed, mark the sample as negative.
14. If a cancer signature is observed, the sample is marked positive, meaning “suspect of having cancer.”
15. Since the process (particularly the CTC identification) is optimized to respond mostly to “organ/tissue of origin” cells, a “suspect of having cancer” implies a “suspect of having cancer from the organ/tissue of interest.” An example of the “cancer in organ/tissue of interest” is “breast cancer.” Alternatively, the existence of tissue-specific marker(s) could imply that the analyzed cells are from the organ/tissue of interest.
16. If for any reason, a sample is qualified as a potential, but fails somewhere in the process, e.g., in the amplification step, the sample is marked as “undetermined” or NoCall. A NoCall sample may be considered for re-processing (obtaining a new blood sample, etc.).

Similar to the SQS, a probabilistic model could apply to the whole (above) process. In this case, in lieu of Detection, a Likelihood number is assigned to the Subject for having the Col. For example, a human Subject may be given a Likelihood of 0.9 (90%) to have cancer. This non-binary call could then be reported to the Subject and/or physician. Alternatively, the non-binary call could be subjected to a threshold, to make a binary call. For example, if the Likelihood is 90% or above, the Col can be labeled as Detected in the Subject.

The unwanted cells are mostly those that belong to different categories of white blood cells (WBCs), but could also belong to cells that belong to other cancers (than the one(s) being interrogated).

The crux of the high specificity (and hence high PPV) in this embodiment is its two-step process, where a sample can be rejected (or called negative) at any of the following two subsystems (in series), each of which is highly specific.
1. SQS: A sample can be marked negative by the Sample Qualification System (SQS), which operates on a relationship built on the antibody staining features of the cells, along with their physical properties such as size.
2. WGS: A sample can be marked as negative (after passing SQS) via not observing the signatures that are expected for breast cancer. Signatures of cancer could be as was stated before, e.g., copy number variations across the genome, as well as somatic single-nucleotide variations (SNVs) and Insertions-Deletions (InDels).

If the specificity of each of these subsystems is only 99%, the combined system would have a specificity of 99.99%.

### Exemplary Depiction of Some Embodiments

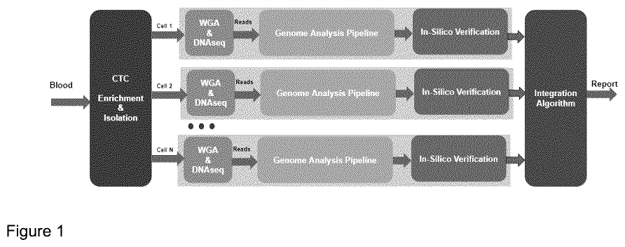

Mutation detection is an integral part of genome analysis for cancer-related applications in both early and late stages. While circulating tumor DNA (ctDNA) assays have gained popularity in recent years, the issues related to their sensitivity, reproducibility, and concordance with tissue have been subjects of much debate. In this work, we show how a novel circulating tumor cell (CTC) based architecture comprising a custom-made genome analysis pipeline could provide a suitable solution for such high demand in variant-detection quality, while providing resilience against sample contamination and several other artifacts attributed to ctDNA. This architecture employs a hybrid-denovo genome analysis pipeline, and artificial intelligence (AI). We argue how, due to lack of standards, an unbiased quality assessment of conventional ctDNA assays has proven to be difficult. This is particularly true when comparing their findings to those of tissue-based assays. This issue could result in complex and, at times, contradicting interpretations. For example, a false positive mutation in ctDNA assay could be hypothesized to be a true positive variant which was missed in the tissue assay due to the local and limited-scope nature of the obtained biopsy from a multi-clonal tumor. Similarly, a false negative in ctDNA could be hypothesized to be a true negative caused by intervention effects, or due to low mutant allele fraction (MAF) in a tumor with an extensive tumor heterogeneity. Sample-to-sample contamination is another concern in ctDNA analysis. This is due to a technical issue in many of the modern DNA sequencers, which could cause a small leakage of sample tags. Although small, the extent of the leakage is large enough to result in contamination levels that are on par with or higher than the actual MAFs of the reported variants. Since these contaminating signals are from another human sample, they have the potential to defy many of the filtering elements in conventional ctDNA analysis methods, and ultimately result in false positives. Another important challenge in this application is that many ctDNA applications focus on detection of a relatively small number of actionable mutations. However, as the scope of these applications has evolved, there has been an increased demand for successful detection and characterization of a more comprehensive set of variants on the genes of interest. Our single-cell architecture (FIG. 1) has the ability to detect variants with a competitive limit of detection (LoD), high detection rate and tissue concordance, along with the power to eliminate sample contamination caused by modern sequencing instruments, and by doing so further improve the specificity.

### DETAILED DESCRIPTION OF FIGURES

### FIG. 1: General Architecture

FIG. 1 shows the general architecture of a proposed single-cell model. Whole blood is subjected to a CTC enrichment and isolation method. Each cell is then lysed to access its genetic material. The DNA of each single cell is subjected to a Nucleotide Amplification (NAM) operation, e.g., whole genome amplification (WGA). While WGA is the preferred mode, NAM could include other methods of amplification, where only a targeted portion of the (and not the entire) genome is amplified, e.g., using Polymerase Chain Reaction (PCR). Throughout this invention, without a loss of generality, the term WGA can be replaced with any generic NAM operation. Among many options for WGA (including home-brew methods), there are many commercial WGA kits [21], [22], [16], [17], [18], e.g., PicoPlex, DOPlify, Repli-g, Ampli-1, GenomePlex, MALBAC, SurePlex. The amplified DNA is then subjected to whole genome sequencing (WGS). After performing WGS, the generated raw reads are presented to Crystal Genetics' genome analysis pipeline, in order to call the underlying variants. Variants related to multiple cells are then integrated in order to make a call set for the genome of interest. In order to reduce the false positive rate, the suspect loci (found by low score, etc.) may be further evaluated via our in-silico visualization (ISV) tool. If the evidence shown by ISV supports the call of interest, it is kept. Otherwise, the call is discarded. ISV can be run prior to or after the integration step. For comparison purposes, a conventional pipeline (for calling variants) can be run and labeled as Benchmark.

Without a loss of generality, the unit of processing could be a pool of cells, instead of a single cell. For example, Cells 1 through N could be replaced with Pools 1 through N. Each pool can have the same, or a different number of cells. For instance, Pool 1 could have 1 cell, Pool 2 could have 5 cells, and Pool 3 could have 2 cells, etc. A pool can have a large number of cells (sometimes referred to as bulk), e.g., a few hundreds, a few thousands or a few millions of cells.

Additionally, a pool can include members of the same species (e.g., the pool of only CTCs, or the pool of only WBCs). A pool can also include members of different species (e.g., a pool of 2 CTCs and 3 WBCs). The members of a pool can be single cells, or cell aggregates/clusters. For instance, a pool can have 1 cluster of CTCs, 2 individual CTCs, and 1 or a few WBCs.

For each locus, the integration of multiple variants (one per each cell or cell pool) to make a single variant (per genome of interest) can be done in different ways. Without a loss of generality, one such example is to accept a variant if at least P percent of the cells agree with the call, e.g., P=50%, P=70%, or P=80%. The calls can be qualified with a predefined Score threshold prior to integration. For example, if a variant call's score was less than S percent, it would be discarded from the tally. Examples of S could be (on a scale of 0 to 1 to mimic the probability of success), 0.5, or 0.7, 0.8, or 0.9. Other variations of filtering prior to, or in the process of integration are allowed.

### FIG. 2: Example Computer System

FIG. 2 illustrates an example computing device or computer system on which some and/or parts of the techniques and methods for high-fidelity condition detection described herein may be implemented. Computer system 100 includes, but is not limited to, one or more processors 102 operationally coupled to memory 106 over one or more buses such as bus 104. Depending on specific implementations and form factors, computer system 100 may also include storage device(s) 108, display device(s) 110, input device(s) 112, and communication device(s) 114.

A processor 102 is a hardware device configured to execute sequences of instructions in order to perform various operations such as, for example, arithmetical, logical, and input/output operations. A typical example of a processor is a central processing unit (CPU), but it is noted that other types of processors such as vector processors and array processors can perform similar operations. Examples of hardware devices that can operate as processors include, but are not limited to, microprocessors, microcontrollers, digital signal processors (DSPs), systems-on-chip, and the like. Processor 102 is configured to receive executable instructions over one or more data and/or address buses such as bus 104. Bus 104 is configured to couple various device components, including memory 106, to processor(s) 102. Bus 104 may include one or more bus structures (e.g., such as a memory bus or memory controller, a peripheral bus, and a local bus) that may have any of a variety of bus architectures. Memory 106 is configured to store data and executable instructions for processor(s) 102. Memory 106 may include volatile and/or non-volatile memory such as read-only memory (ROM) and random-access memory (RAM). For example, a basic input/output system (BIOS) containing the basic executable instructions for transferring information between system components (e.g., during start-up) is typically stored in ROM. RAM typically stores data and executable instructions that are immediately accessible and/or being operated on by processor(s) 102 during execution. Memory 106 is an example of a non-transitory computer-readable medium.

Computer-readable media may include any available medium that can be accessed by a computer system (and/or the processors thereof) and includes both volatile and non-volatile media and removable and non-removable media. One example of non-transitory computer-readable media is storage media. Storage media includes media implemented in any method or technology for storage of information such as computer-readable instructions, data structures, program modules, and/or other data. Examples of storage media include, but are not limited to, RAM, ROM, electrically erasable programmable read-only memory (EEPROM), removable memory such as flash memory and solid state drives (SSD), compact-disk read-only memory (CD-ROM), digital versatile disks (DVD) and other optical disks, magnetic cassettes, magnetic tapes, magnetic disks or other magnetic storage devices, electromagnetic disks, and any other medium which can be used to store the desired information and which can be accessed and read by a computer system. Another example of computer-readable media is communication media. Communication media typically embody computer-readable instructions, data structures, program modules, or other data, in a modulated data signal such as a carrier wave or other transport mechanism and includes any information delivery media. By way of example, and not limitation, communication media includes wired media such as a wired network or direct-wired connection, and wireless media such as acoustic, radio frequency (RF), infrared and other wireless media.

Computer system 100 may include, and/or have access to, various non-transitory computer-readable media that is embodied in one or more storage devices 108. Storage device(s) 108 may be coupled to processors(s) 102 over one or more buses such as bus 104. Storage device(s) 108 are configured to provide persistent storage of executable and other computer-readable instructions, data structures, program modules, and other data for computer system 100 and/or for its users. In various embodiments and form factors of computer system 100, storage device(s) 108 may include persistent storage media of one or more types including, but not limited to, electromagnetic disks (e.g., hard disks), optical storage disks (e.g., DVDs and CD-ROMs), magneto-optical storage disks, solid-state drives, flash memory cards, universal serial bus (USB) flash drives, and the like. By way of example, storage device(s) 108 may include a hard disk drive that stores the executable instructions of an Operating System (OS) for computer system 100, the executable instructions of one or more computer programs, clients, and other computer processes that can be executed on the computer system, and any OS and/or user data in various formats.

Computer system 100 may also include one or more display devices 110 and one or more input devices 112 that are coupled to processor(s) 102 over one or more buses such as bus 104. Display device(s) 110 may include any devices configured to receive information from, and/or present information to, user(s) of computer system 100. Examples of such display devices include, but are not limited to, cathode-ray tube (CRT) monitors, liquid crystal displays (LCDs), light emitting diode (LED) displays, field emission (FED, or “flat panel” CRT) displays, plasma displays, electro-luminescent displays, and any other types of display devices. Input device(s) 112 may include a general pointing device (e.g., such as a computer mouse, a trackpad, or an equivalent spatial-input device), an alphanumeric input device (e.g., such as a keyboard), and/or any other suitable human interface device (HID) that can communicate commands and other user-generated information to processor(s) 102.

Computer system 100 may include one or more communication devices 114 that are coupled to processor(s) 102 over one or more buses such as bus 104. Communication device(s) 114 are configured to receive and transmit data from and to other devices and computer systems. For example, communication device(s) 114 may include one or more USB controllers for communicating with USB peripheral devices, one or more network storage controllers for communicating with storage area network (SAN) devices and/or network-attached storage (NAS) devices, one or more network interface cards (NICs) for communicating over wired communication networks, and/or one or more wireless network cards for communicating over a variety of wireless data-transmission protocols such as, for example, IEEE 802.11 and/or Bluetooth. Using communication device(s) 114, computer system 100 may operate in a networked environment using logical and/or physical connections to one or more remote computer systems and/or other computing devices. For example, computer system 100 may be connected to one or more remote computers that provide access to block-level data storage over a SAN protocol and/or to file-level data storage over a NAS protocol. In another example, computer system 100 may be connected to one or more networks 116 over connections that support one or more networking protocols. Network(s) 116 may include, without limitation, a local area network (LAN), a wide area network (WAN), a global network (e.g., the Internet), and/or any other type of network or combination of networks. Some embodiments and/or parts of the techniques for high-fidelity condition detection described herein may be implemented as a computer program product that may include sequences of instructions stored on non-transitory computer-readable media. These instructions may be used to program one or more computer systems that include one or more special-purpose or general-purpose processors (e.g., CPUs) or equivalents thereof (e.g., such as processing engines, processing cores, etc.). When executed by the processor(s), the sequences of instructions cause the computer system(s) to perform the operations according to some of the embodiments of the techniques described herein. Additionally, or instead of, some embodiments of the techniques described herein may be practiced in distributed computing environments that may involve more than one computer system. One example of a distributed computing environment is a client-server environment, in which some of the various functions of the techniques described herein may be performed by a client program product executing on a computer system and some of the functions may be performed by a server program product executing on a server computer. Another example of a distributed computing environment is a cloud computing environment. In a cloud computing environment, computing resources are provided and delivered as a service over a network such as a local-area network (e.g., LAN) or a wide-area network (e.g., the Internet). Examples of cloud-based computing resources may include, without limitation: physical infrastructure resources (e.g., physical computing devices or computer systems, and virtual machines executing thereon) that are allocated on-demand to perform particular tasks and functions; platform infrastructure resources (e.g., an OS, programming language execution environments, database servers, web servers, etc.) that are installed/imaged on-demand onto the allocated physical infrastructure resources; and application software resources (e.g., application servers, single-tenant and multi-tenant software platforms, etc.) that are instantiated and executed on-demand in the environment provided by the platform infrastructure resources. Another example of a distributed computing environment is a computing cluster environment, in which multiple computing devices each with its own OS instance are connected over a fast local network. Another example of a distributed computing environment is a grid computing environment in which multiple, possibly heterogeneous and/or geographically dispersed, computing devices are connected over conventional network(s) to perform a common task or goal. In various distributed computing environments, the information transferred between the various computing devices may be pulled or pushed across the transmission medium that connects the computing devices.

### FIG. 3: Example DNA Sequencing System

FIG. 3 illustrates an example DNA sequencing system 200 on which some and/or parts of the techniques and methods described herein may be implemented. In some embodiments, DNA sequencing system 200 may be a high throughput instrument capable of sequencing oligos by using any suitable next generation sequencing (NGS) technology. Examples of such DNA sequencing systems include, without limitation, the MiSeq, HiSeq, NextSeq, iSeq and NovaSeq sequencers manufactured by Illumina, Inc., lon Proton systems manufactured by Life Technologies, Inc., BGIseq sequencers manufactured by BGI (designed by Complete Genomics, Inc.), or MinION/PromethION sequencers manufactured by Oxford Nanopore Technologies. It is noted, however, that various other DNA sequencing systems available on the market may be suitable for implementing the techniques described herein.

DNA sequencing system 200 includes a sequencing device (sequencer) 202 that is communicatively and/or operatively coupled to computer system 220. Sequencer 202 includes compartments that can accept flow cell(s) or slides 204 with the oligos being sequenced (target oligos), cartridge(s) 206 with the sequencing reagents and buffers used during sequencing, and detection instrument 208 which performs the sequencing. According to the techniques and methods described herein, the target oligos may represent full or partial genomes and/or mixtures thereof. Various fluidic lines, tubing, valves, and other fluidic connections may be used to connect the compartments with flow cell(s) or slides 204 and cartridge(s) 206 to detection instrument 208. A flow cell 204 may include a housing that encloses a solid support (e.g., a microarray, a chip, beads, etc.), with one or more ports being provided for loading the target oligos into the flow cell and for administering the various reagents and buffers during sequencing cycles. In some sequencing systems, the target oligos may be pre-processed into libraries by applying various chemical steps such as denaturing, diluting, etc. A cartridge 206 is used to store various sequencing reagents, buffers, chemicals, as well as any waste that are needed or produced during sequencing. For example, a cartridge 206 may include suitable storage reservoirs that store denaturation agents (e.g., formamide), wash solutions, probes, etc.

Detection instrument 208 is configured to detect the DNA sequences of the target oligos and to generate reads 209. In various embodiments, detection instrument 208 may utilize various sequencing mechanisms such as, for example, sequencing by synthesis, sequencing by ligation, sequencing by hybridization, etc., where such mechanisms may be employed in massively-parallel fashion in order to increase throughput. Further, in various embodiments detection instrument 208 may detect the DNA bases of the target oligos by using optical-based detection, semiconductor-based (or electronic) detection, electrical-based (e.g., nanopore) detection, etc. In various embodiments, detection instrument 208 may also include various suitable mechanical and/or electro-mechanical components that may be configured to position the flow cell 204 at the beginning and/or during sequencing.

Computer system 220 is a suitable computing device and may be communicatively coupled to a network 216. Examples of such computer systems and networks are described above with respect to FIG. 2. Referring to FIG. 3, computer system 220 is configured to execute software programs that control the operation of sequencer 202 to generate the reads 209 that represent the DNA sequences of the target oligos, in accordance with the techniques described herein. For example, computer system 220 may be configured with suitable software program(s) or application(s) that control the various sequencing cycles performed by sequencer 202. In addition, in some embodiments computer system 220 may be further configured to perform various post-sequencing steps in accordance with the techniques described herein such as, for example, performing error correction on reads 209, assembling longer reads from the generated reads 209, etc.

In operation, computer system 220 controls the operation of DNA sequencing system 200. Sequencing system 200 is first loaded with flow cell(s) or slides 204 that contain the target oligos and with the sequencing cartridge(s) 206. Prior to and/or after loading the flow cells/slides, the target oligos may be amplified (e.g., by using polymerase chain reaction, PCR) in order to preserve a sufficient amount for each read. Then the system performs its sequencing cycles and generates sequencing reads 209 that represent the DNA sequences of the target oligos. A read is generally a sequence of data values that represent (fully or partially) the DNA sequence of a corresponding target oligo. According to the techniques described herein, computer system 220 and the software executing thereon control then perform the methods described herein.

### REFERENCES

[1] https://patents.google.com/patent/US20160273047A1/en
[2] https://www.ncbi.nlm.nih.gov/pmc/articles/PMC5652773/[3]
[3] https://www.sciencedirect.com/science/article/pii/S0169409X18300024
[4] http://europepmc.org/abstract/MED/25358515
[5] https://www.ncbi.nlm.nih.gov/pubmed/12459625
[6] https://www.ncbi.nlm.nih.gov/pmc/articles/PMC4934037/[7]
[7] https://www.ncbi.nlm.nih.gov/pmc/articles/PMC5836084/[8]
[8] https://patentimages.storage.googleapis.com/77/f5/0b/3c0dbd463114d0/US201602730 47A1.pdf
[9] https://www.karger.com/Article/Pdf/487571
[10] https://www.ncbi.nlm.nih.gov/pmc/articles/PMC4975257/
[11] http://clinchem.aaccjnls.org/content/58/5/936
[12] https://www.ncbi.nlm.nih.gov/pubmed/28168712
[13] https://link.springer.com/protocol/10.1007% 2F978-1-4939-3444-7_9
[14] http://journals.plos.org/plosone/article?id=10.1371/journal.pone.0171566
[15] Kamal et al. https://www.nature.com/articles/s41598-020-60008-3.pdf
[16] Deleye, Coninck, et al. https://www.nature.com/articles/srep11711.pdf
[17] Chen et al. https://www.nature.com/articles/s41598-018-23325-2.pdf
[18] Carretta et al. https://www.mdpi.com/1422-0067/21/19/7366/htm
[19] https://www.ncbi.nlm.nih.gov/pmc/articles/PMC5070309/https://www.nature.com/articles/aps201817
[20] https://www.nature.com/articles/aps201817
[21] Deleye et al. https://www.nature.com/articles/s41598-017-03711-y.pdf
[22] Zhang et al. https://www.ncbi.nlm.nih.gov/pmc/articles/PMC6434089/pdf/bsr-37-bsr20170252.pdf
[23] https://www.cellsearchctc.com/product-systems-overview/cellsave-preservative-tubes
[24] https://www.stemcell.com/
[25] https://www.pluriselect.com/us/
[26] Sharma et al., PMID: 29559380 https://pubmed.ncbi.nlm.nih.gov/29559380/
[27] Ferreira, Ramani, Jeffrey https://www.sciencedirect.com/science/article/pii/S157478911600017X
[28] Habli et al., Cancers 2020, 12, 1930; doi: 10.3390/cancers12071930 https://www.mdpi.com/2072-6694/12/7/1930/pdf
[29] https://www.beckman.com
[30] https://www.thermofisher.com
[31] Sato et al. doi: 10.1093/dnares/dsz017
[32] https://www.covaris.com
[33] https://www.agilent.com

What is claimed is:

1. A method for Detection of a Condition of Interest (Col) in a Subject, said method comprising the steps of:
obtaining a Biological Sample from said Subject; and
performing Enrichment operation on at least a portion of the Biological Sample for the Features of Interest (FoI) to arrive at the Enriched Biological Sample (EBS); and
feeding at least a portion of the EBS to a Sample Qualification System (SQS) to label the Biological Sample as Likely (Qualified) or Not Likely (not Qualified) in relation to said Col; and
passing at least a portion of the said Qualified Samples to the Feature Isolation Step (FIS) to arrive at the Isolated Cells; and
Subjecting at least a portion the Isolated Cells to Feature Molecular Analysis (FMA); and
declaring the state of the Subject as Detected for the said Col if the FMA results in a positive call.

2. A method as set forth in claim 1, wherein said Col is cancer.

3. A method as set forth in claim 1, wherein said Subject is human.

4. A method as set forth in claim 1, wherein said Biological Sample is Blood.

5. A method as set forth in claim 1, wherein said Enrichment comprises Positive Selection.

6. A method as set forth in claim 1, wherein said Enrichment comprises Negative Selection.

7. A method as set forth in claim 1, wherein said Features of Interest comprise cells with characteristics related to the said Col.

8. A method as set forth in claim 1, wherein said FoI comprise at least one Circulating Tumor Cell (CTC).

9. A method as set forth in claim 1, wherein said FoI comprise at least one container with a single cell.

10. A method as set forth in claim 1, wherein said FoI comprise at least one container with a pool of cells.

11. A method as set forth in claim 1, wherein said SQS uses at least one of the Physical Properties of said Fol.

12. A method as set forth in claim 1, wherein said SQS uses at least one Affinity Based Method for said Fol.

13. A method as set forth in claim 1, wherein said FIS comprises Physical Picking of the cells of interest, to arrive at said Isolated Cells.

14. A method as set forth in claim 1, wherein said FIS comprises Informatic Isolation of the cells of interest, to arrive at said Isolated Cells.

15. A method as set forth in claim 1, wherein said FIS comprises using a Positive Selection Method.

16. A method as set forth in claim 1, wherein said FIS comprises using a Negative Selection Method.

17. A method as set forth in claim 1, wherein said FMA comprises a Nucleotide Amplification (NAM) operation.

18. A method as set forth in claim 1, wherein said FMA comprises Nucleic Acid Analysis (NAA).

19. A method for Detection of a Condition of Interest (Col) in a Subject, said method comprising the steps of:
obtaining a Biological Sample from said Subject; and
performing Enrichment operation on at least a portion of the Biological Sample for the Features of Interest (FoI) to arrive at the Enriched Biological Sample (EBS); and
feeding at least a portion of the EBS to a Sample Qualification System (SQS) to assign a non-binary Likelihood to the Qualification of Biological Sample in relation to said Col; and
passing at least a portion of the said Sample with high Likelihood to the Feature Isolation Step (FIS) to arrive at the Isolated Cells; and
Subjecting at least a portion the Isolated Cells to Feature Molecular Analysis (FMA); and
declaring the state of the Subject as Detected for the said Col if the FMA results in a positive call.

20. A method for Detection of a Condition of Interest (Col) in a Subject, said method comprising the steps of:
obtaining a Biological Sample from said Subject; and
performing Enrichment operation on at least a portion of the Biological Sample for the Features of Interest (FoI) to arrive at the Enriched Biological Sample (EBS); and
feeding at least a portion of the EBS to a Sample Qualification System (SQS) to assign a non-binary Likelihood to the Qualification of Biological Sample in relation to said Col; and
passing at least a portion of the said Sample to the Feature Isolation Step (FIS) to arrive at the Isolated Cells; and
subjecting at least a portion the Isolated Cells to Feature Molecular Analysis (FMA) to arrive at a Likelihood based on FMA, and
calculate an overall Likelihood, based on the Likelihoods defined in SQS and FMA, for the said Subject to be related to the said Col.
